(19) United States
(12) Patent Application Publication
(10) Pub. No.: US 2026/0345381 A1
(43) Pub. Date: Nov. 5, 2026

(54) BOTTLE ADAPTOR AND METHODS OF USING SAME

(71) Applicant: The Geneva Foundation, Tacoma, WA (US)

(72) Inventors: Kevin JACKSON, Tacoma, WA (US); Nicholas A. CHARTRAIN, Tacoma, WA (US); Emily WISDOM, Tacoma, WA (US); Vincent B. HO, Tacoma, WA (US)

(73) Assignee: The Geneva Foundation, Tacoma, WA (US)

(21) Appl. No.: 19/486,499

(22) PCT Filed: May 23, 2024
(86) PCT No.: PCT/US2024/030703

§ 371 Date: Nov. 21, 2025

Related U.S. Application Data
(60) Provisional application No. 63/468,618, filed on May 24, 2023.

(51) Int. Cl. A61F 9/00; A61M 3/02

(52) U.S. Cl. CPC A61F 9/0026; A61M 3/0245; A61M 3/0279; A61M 2210/0612

(57) ABSTRACT

The disclosure relates to an adaptor including a body configured to be at least partially inserted into an open end of a bottle while forming a seal with an inner peripheral surface of the open end of the bottle, systems for use in delivering a liquid for treatment of an individual including the adaptor, and methods for irrigation of an eye using the adaptor.

### PRIORITY

This application claims priority under 35 U.S.C. § 119(e) to U.S. Provisional Application No. 63/468,618, filed May 24, 2023, the contents of which are incorporated herein by reference in their entirety.

### GOVERNMENT SUPPORT

This invention made with government support under Contract No. HU00011920022 awarded by USAMRDC CDMRP/Uniform Services University. The government has certain rights in the invention.

### KEYWORDS

Chemical injury, eye, lavage, water bottle, on the go, IV tubing

### INTRODUCTION

Eye injuries are common in warfare. Chemical burns to the eye are among the most urgent of ocular emergencies and the extent of ocular damage and visual impairment are minimized with rapid initiation of irrigation. Ocular irrigation is a well-known procedure for treatment of eyes that are traumatized or otherwise have medical ailments. However, effective ocular irrigation options are often not available in resource-constrained environments. An ocular lens, as disclosed in U.S. Pat. No. 3,664,340 (“the '340 patent”) is a commonly used irrigation device that provides continuous lavage to the cornea by directing fluid to contact-lens like inserts which rest on the eyes. The ocular lens of the '340 patent may be used for emergency ocular irrigations using fluid delivered from any disposable water bottle. The widespread availability of disposable water bottles, even in resource-constrained environments, and the small size of the disclosed adaptor and the ocular lens of the '340 patent will allow effective ocular irrigation in austere environments at or near the point of injury. While the standard of care in hospital settings uses specialized irrigation fluids, bottled water is a suitable alternative in emergency situations.

Rapid prototyping and design iteration of the novel device was enabled through the use of Additive Manufacturing (3D printing). 3D printing has enabled point of need fabrication using a variety of materials and has allowed for the rapid deployment and implementation of solutions in austere operating environments. The disclosed adapter is an example of a novel solution to a clinical problem enabled by 3D printing coupled with the combined expertise of clinicians and design engineers. These additive manufacturing solutions can be used to develop devices to overcome the specific challenges faced by service members in battlefield environments.

The ocular lens of the '340 patent, however, is limited to use with availability of IV bags and can be difficult to employ in resource-constrained, austere, or mass casualty environments where insufficient amounts of resources exist. This disclosure introduces a universal adaptor that facilitates emergency ocular irrigation in austere environments.

### SUMMARY OF THE DISCLOSURE

In a first aspect of the disclosure, an exemplary adaptor may include a resilient, frustoconical body configured to be at least partially inserted into an opening at one end of a bottle while forming a seal with an inner peripheral surface of the opening. The body may define first and second passageways extending through the body from an upper surface of the body to a lower surface of the body. The first passageway may have a generally square-shaped cross-section along at least a portion of the length of the first passageway. The second passageway may have a generally circular-shaped cross-section along at least a portion of the length of the second passageway.

In a second aspect of the disclosure, a method for irrigation of an eye may include inserting a body of an adaptor into an open end of a bottle. The adaptor may include a resilient, frustoconical body configured to be at least partially inserted into the open end of the bottle while forming a seal with an inner peripheral surface of the open end of the bottle. The method may further include inserting a venting conduit and tubing into the adaptor. The method may still further include connecting the tubing to an ocular lens configured for irrigation of the eye, and flowing liquid from the bottle into the tubing, and irrigating the eye via the ocular lens.

### BRIEF DESCRIPTION OF THE DRAWINGS

FIGS. 1A-B depict views from opposite sides of the disclosed adaptor, showing passageways with circular and rectangular cross-sections.

FIG. 2 is a cross-sectional view along line 2-2 in FIG. 1B of an exemplary embodiment of the disclosed adaptor with square-shaped and circular-shaped passageways running along the length of the adaptor.

FIG. 3 depicts an exemplary embodiment of the disclosed adaptor attached to a bottle with a venting conduit attached to the adaptor and protruding into the bottle and one end of the venting conduit projecting out from the adaptor.

FIG. 4 depicts an exemplary embodiment of the disclosed adaptor with a circular cross-section tube and a rectangular cross-section venting conduit penetrating the adaptor and with the venting conduit protruding into the bottle.

FIG. 5 depicts an exemplary embodiment of the disclosed venting conduit.

FIG. 6 depicts an exemplary embodiment of the disclosed venting conduit with frangible portions spaced apart from one another.

FIG. 7 is a cross-sectional view along line 7-7 in FIG. 5 of the disclosed venting conduit.

FIG. 8 is a cross-sectional view along line 8-8 in FIG. 6 of the disclosed venting conduit with frangible portions spaced apart from one another.

FIG. 9 depicts an exemplary embodiment of the disclosed adaptor connecting a bottle to tubing with a venting conduit attached to the adaptor and protruding into the bottle and with the tubing connected to an ocular lens.

FIG. 10 depicts an exemplary embodiment of the disclosed adaptor connecting a bottle to tubing with a venting conduit attached to the adaptor and protruding into the bottle and with the tubing connected to an ocular lens in close proximity to a patient's eye.

### DETAILED DESCRIPTION

Outside of the hospital, availability of IV bags to enable ideal long-duration, steady lavage of a wound or eye is basically non-existent for practical reasons. Water bottles, however, are ubiquitous. Although non-sterile, these sources are ideal when connected to IV tubing to provide slow steady lavage. An adaptor according to various embodiments described in this disclosure may enable the use of water bottles in place of IV bags for the containment of liquids used during lavage of a wound or an eye.

In an exemplary method according to various embodiments of this disclosure, the cap may be removed from a typical water bottle, and an adaptor according to various embodiments of this disclosure may be inserted into the open end of the bottle to seal the bottle. An exemplary embodiment of the adaptor may accommodate a venting conduit that extends through a first passageway penetrating through the adaptor and that protrudes into the bottle to allow for equalization of atmospheric air pressure as liquid flows from the bottle, thus enabling the steady flow of the liquid from the bottle. An exemplary embodiment of the adaptor may also accommodate IV tubing that extends at least part way into a second passageway penetrating through the adaptor. Liquid, including various pharmaceutical solutions contained within the bottle, may flow smoothly from the bottle and through the IV tubing to an application area on a patient. Water bottle tops may have many different threaded configurations such that a large variety of different threaded bottle caps would be needed to properly cap a bottle. The disclosed adaptor may be composed of a resilient material and may be tapered such that a single exemplary configuration of the adaptor may be used to seal closed different openings in a large variety of different water bottles, and provide a stable connection for venting conduit and/or tubing that allows for the dispensing of liquid from the bottles. Various disclosed embodiments of the adaptor according to this disclosure may be configured to accommodate a venting conduit that penetrates through the adaptor and extends into the bottle for the purpose of equalizing atmospheric air pressure to allow for smooth dispensation of liquid from the bottle. Embodiments of the adaptor may also accommodate IV tubing that may be inserted at least part way into a passageway extending through the adaptor for enabling the flow of liquid from the bottle. The passageways through the adaptor for accommodating a venting conduit and the IV tubing may be provided with different cross-sectional configurations and dimensions, depending on a variety of different factors that may include the viscosity or other characteristics of a liquid to be dispensed from the bottle, a desired rate of flow of the liquid from the bottle, temperatures and other environmental considerations where the adaptor is to be used, and manufacturability considerations, such as the ease in producing the adaptor using additive manufacturing techniques such as 3D printing.

In an exemplary embodiment of this disclosure, the adaptor may include a resilient, frustoconical body configured to be at least partially inserted into an open end of a bottle while forming a seal with an inner peripheral surface of the opening into the bottle. The body of the adaptor may define first and second passageways extending through the body. The first passageway may have a respective opening in each of an upper surface of the body and a lower surface of the body at opposite ends of the first passageway, and a generally square-shaped cross-section along at least a portion of the length of the first passageway. The second passageway may have a respective opening in each of the upper surface of the body and the lower surface of the body at opposite ends of the second passageway, and a generally circular-shaped cross-section along at least a portion of the length of the second passageway.

In some embodiments, the adaptor may be frustoconical in shape, and one or both of the first passageway and the second passageway may be tapered along the length of the adaptor such that the respective openings at opposite ends of one or both of the passageways may have different dimensions.

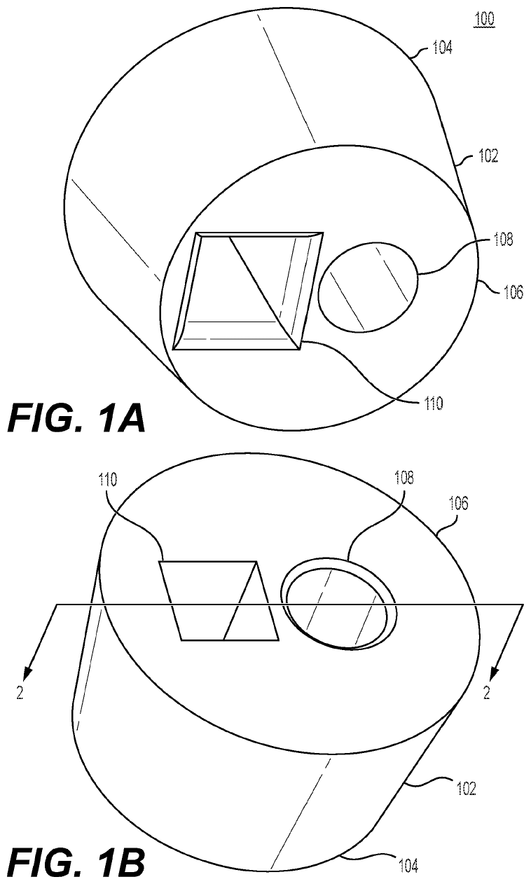

As shown in FIGS. 1A and 1B, an exemplary disclosed adaptor 100 may include a body 102 and two faces 104, 106 at opposite ends of the body 102, with each face including a circular orifice 108 and a square or rectangular orifice 110.

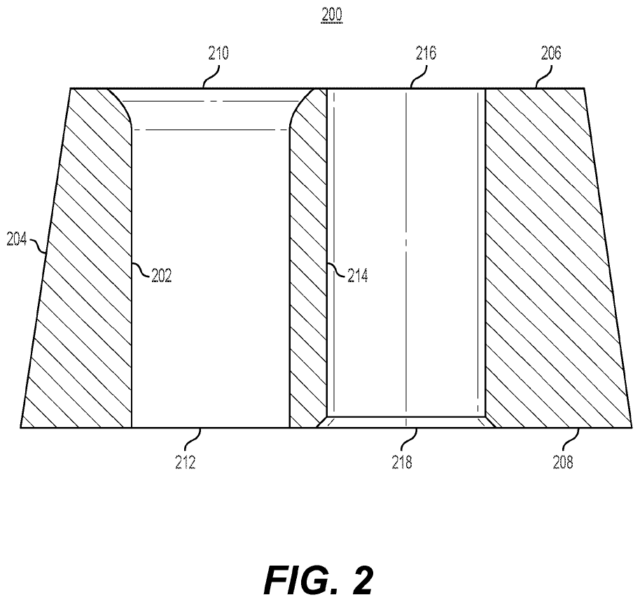

As shown in FIG. 2, an exemplary disclosed adaptor 200 may include a passageway 202 with a circular cross-section penetrating through the length of the adaptor body 204 from a first face 206 of the adaptor 200 to a second face 208 at an opposite end of the adaptor body 204. The passageway 202 may terminate in a circular orifice 210 at the first face 206 and a separate, circular orifice 212 at the second face 208. The exemplary adaptor 200 may additionally include a passageway 214 with a square or rectangular cross-section penetrating through the length of the adaptor body 204 and terminating in a square or rectangular orifice 216 on the first face 206 of the adaptor 200 and a separate, square or rectangular orifice 218 on its second face 208. The dimensions of the orifices at the opposite ends of each of the passageways 202, 214 may be the same in embodiments wherein each of the passageways has the same cross-sectional area along its length. Alternatively, the dimensions of the orifices at the opposite ends of each of the passageways may be differ from one another when the passageways are tapered along their lengths, or when the openings into the passageways are chamfered, flared, or otherwise configured.

In other embodiments, the adaptor body may be shaped and configured differently. For example, the adaptor body may be tapered, cone-shaped, or truncated, or some combination thereof. In some embodiments, the adaptor body may define more than two passageways extending through the body, such as, for example, three, four, or five passageways. In some embodiments, the first passageway may be configured or shaped differently. For example, the first passageway may be circular-shaped or triangular-shaped. In some embodiments, the second passageway may be configured or shaped differently. For example, the second passageway may be square-shaped or triangular-shaped.

In some embodiments, the adaptor body may be made of thermoplastic polyurethane. Alternatively, the adaptor body may be made of some other functional material.

In some embodiments, the adaptor body may be configured to receive a venting conduit and tubing, respectively. Alternatively, the adaptor body may be configured to receive any material or feature that can fit in its orifices and passageways.

A further exemplary embodiment of the disclosure may provide a system for use in delivering a liquid for treatment of an individual. The system may comprise the disclosed adaptor, the disclosed venting conduit, and the disclosed tubing. The venting conduit may have a generally rectangular cross-section along at least a portion of the length of the venting conduit. The venting conduit may have a plurality of frangible portions spaced apart from one another along the length of the venting conduit. The frangible portions may be configured to permit a portion of the venting conduit to be removed from a remainder of the venting conduit so as to shorten the length of the venting conduit. In alternative embodiments, the venting conduit may be shaped differently, such as with a circular or triangular cross-section, and/or may not contain frangible portions. In some embodiments, the tubing may be employed for irrigation. Alternatively, the tubing may be employed for any other applicable purpose.

In some embodiments, the system may comprise a bottle. The bottle may contain a liquid used for ocular irrigation for treatment of an eye. In alternative embodiments, the bottle may be employed for any other purpose. In some embodiments, the venting conduit may extend through the above-disclosed first passageway and protrude into the bottle such that an end of the venting conduit is proximate to a closed end of the bottle opposite from the open end of the bottle. The venting conduit enables equalization of atmospheric air pressure as liquid is dispensed from the bottle, thus preventing a vacuum from forming at the bottom of the bottle when liquid is discharged from the bottle. The venting conduit may penetrate through the adaptor according to various embodiments of this disclosure and extend into the bottle to a point near the closed bottom of the bottle in order to allow external atmospheric air to be sent directly to the bottom of the bottle as the liquid begins to flow from the bottle.

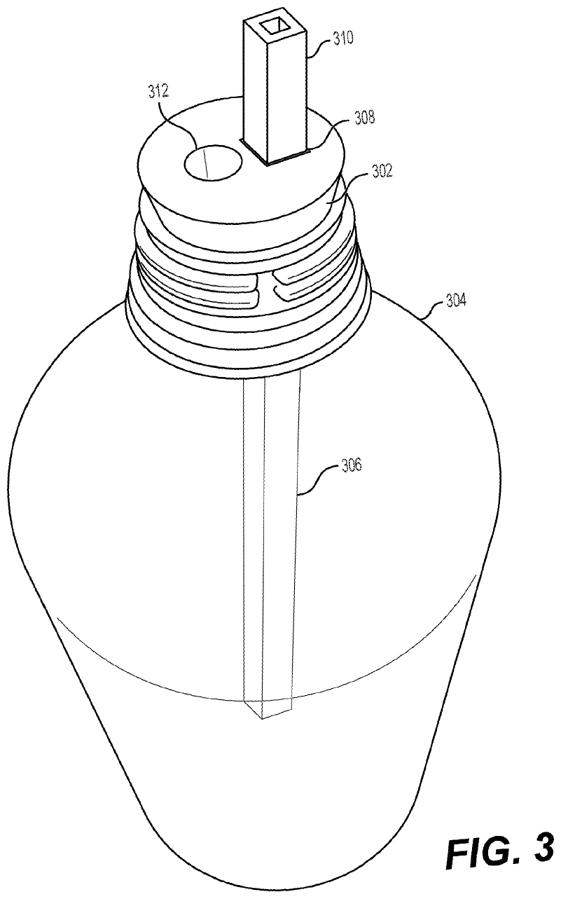

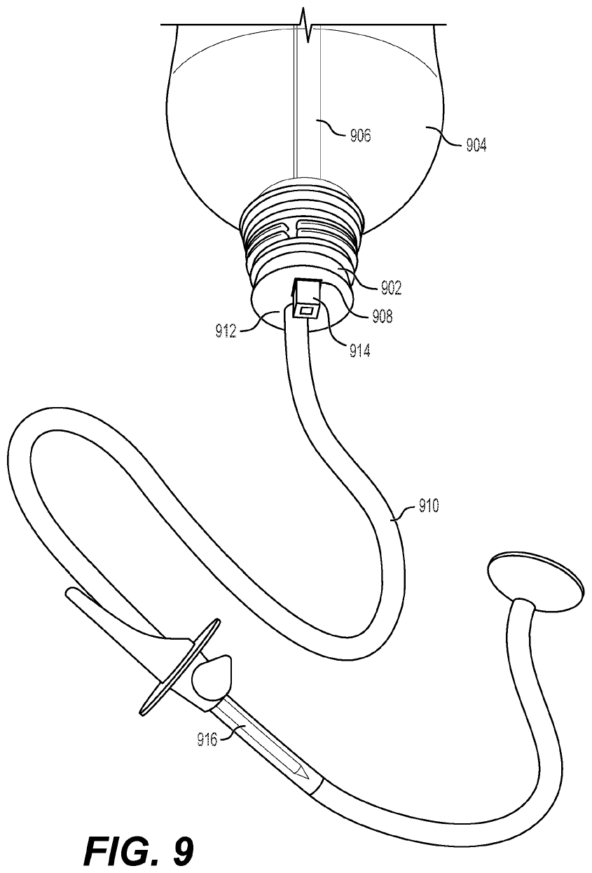

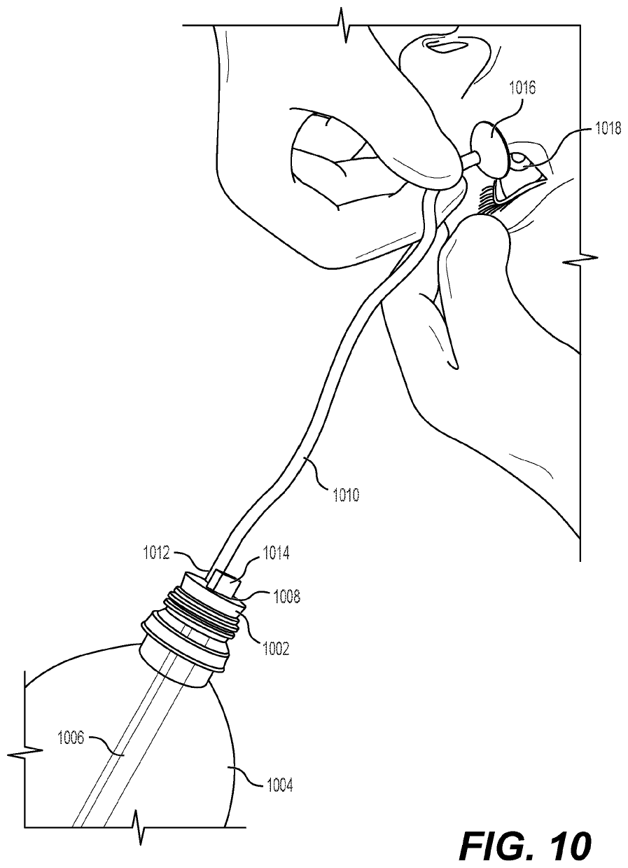

As shown in the exemplary embodiment of FIG. 3, an adaptor body 302 may cap a bottle 304 with a square-shaped venting conduit 306 penetrating the adaptor body 302 at a square orifice 308 and extending into the bottle 304. One end 310 of the venting conduit 306 may project out from the adaptor body 302. In other embodiments, the venting conduit 306 may terminate flush with an upper surface of the adaptor body 302. The adaptor body 302 may also include a circular orifice 312, adjacent to the square orifice 308, and defining an opening to a second passageway penetrating through the adaptor body for accommodating tubing, as shown in FIGS. 9 and 10.

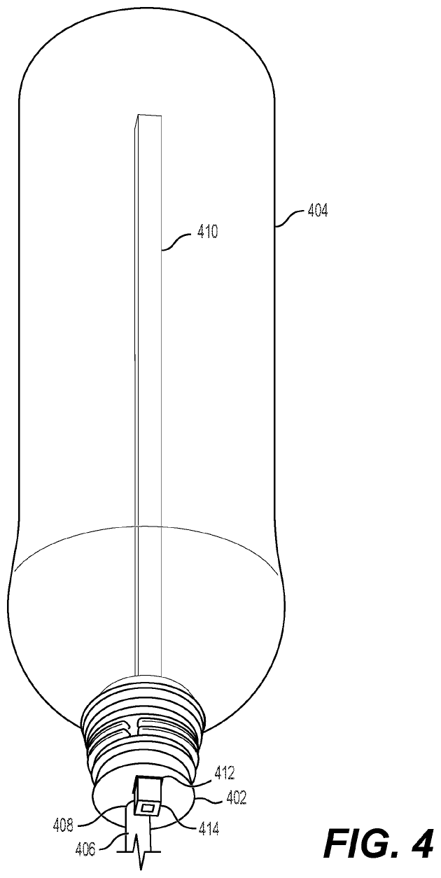

As shown in an exemplary embodiment of FIG. 4, an adaptor body 402 may cap a bottle 404, and may include a circular orifice 408 at an opening to a passageway through the adaptor body 402 for accommodating tubing 406 (having, e.g., a circular cross-section) that penetrates the adaptor body 402 to allow for dispensation of liquid from the bottle 404. The adaptor body 402 may also include a square orifice 412 at an opening to another passageway through the adaptor body 402 for accommodating venting conduit 410 (having, e.g., a square-shaped cross-section) that penetrates the adaptor body 402 and extends into the bottle 404. One end 414 of the venting conduit 410 may project out from the adaptor, or, in alternative embodiments, may be disposed flush with the top surface of the adaptor body 402.

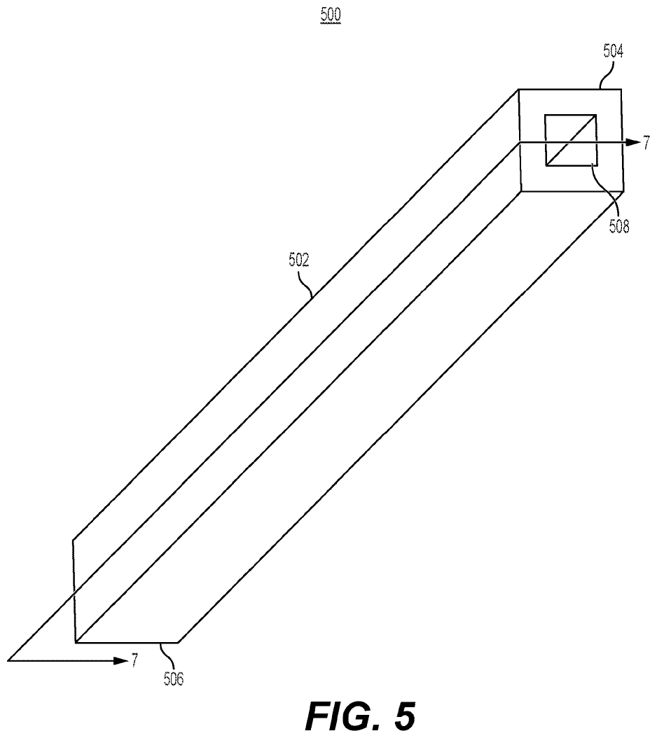

As shown in FIG. 5, an embodiment of a venting conduit 500 may include a body 502 and two end faces 504, 506 disposed at opposite ends of the body 502, with a square orifice 508 defined at each of opposite ends of a square cross-section passageway extending through the venting conduit 500.

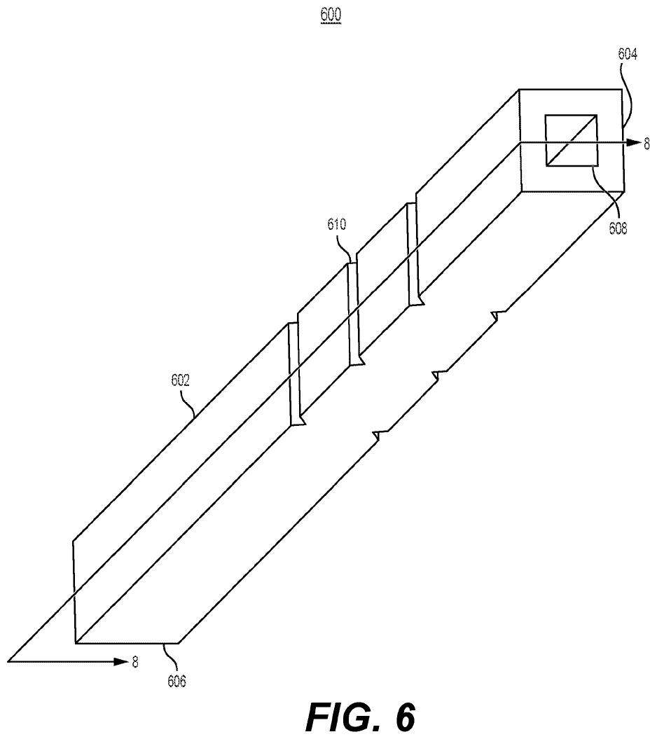

As shown in FIG. 6, another embodiment of a venting conduit 600 may include a body 602 and two end faces 604, 606 disposed at opposite ends of the body 602, with a square orifice 608 defined at each of opposite ends of a square cross-section passageway extending through the venting conduit 600. One or more frangible portions 610 may be formed at various points along the length of the body 602 in order to facilitate breakage and removal of portions of the venting conduit 600 so as to shorten its length.

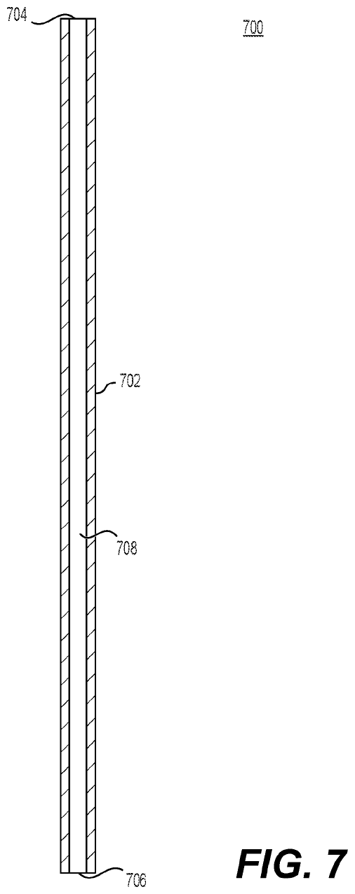

FIG. 7 illustrates a cross-sectional view, such as a view taken along line 7-7 in FIG. 5, showing another venting conduit 700, which may include a body 702 and two end faces 704, 706 disposed at opposite ends of the body 702, with a passageway 708 extending through the length of the body 702. In various alternative configurations, the passageway 708 may have various cross-sectional configurations, such as square, rectangular, circular, triangular, and polygonal.

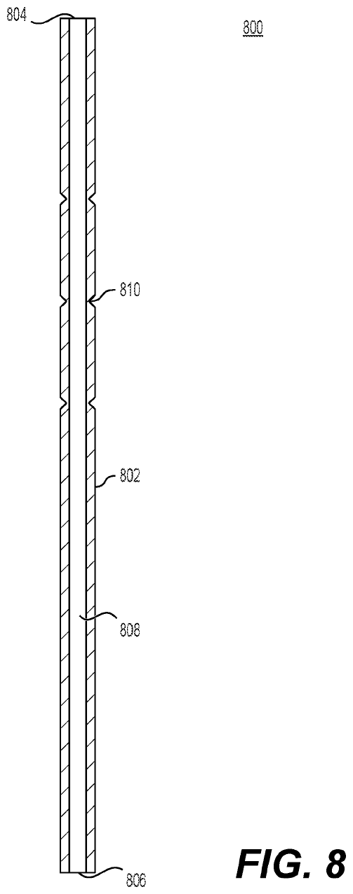

FIG. 8 illustrates a cross-sectional view, such as a view taken along line 8-8 in FIG. 6, showing yet another venting conduit 800, which may include a body 802 and two end faces 804, 806 disposed at opposite ends of the body 802, with a passageway 808 extending through the length of the body 802 and one or more frangible portions 810 defined at various points along the length of the body 802 in order to facilitate breakage and removal of portions of the venting conduit 800 so as to shorten its length.

In some embodiments, the disclosed tubing may include a spike configured to extend through the above-disclosed second passageway. In further embodiments, the disclosed venting conduit may be made of polyethylene terephthalate glycol. Alternatively, the venting conduit may be made of some other functional material. In further embodiments, the disclosed tubing may be connected to an ocular lens configured to irrigate an eye. In certain embodiments, the ocular lens may be cup-shaped. The ocular lens may define an inner portion configured to cover a cornea and an outer portion defining a peripheral rim. In alternative embodiments, the ocular lens may be shaped differently, as long as it remains configured to cover a subject's eye.

As shown in FIG. 9, the adaptor body 902 may cap a bottle 904 with a square-shaped venting conduit 906 extending into the bottle 904 and connected to the square orifice 908 of the adaptor body 902 and tubing 910 connected to the circular orifice 912 of the adaptor body 902. One end 914 of the venting conduit 906 may project out from the adaptor. The tubing 910 may include a tubing spike 916, which facilitates irrigation.

As shown in FIG. 10, the adaptor body 1002 may cap a bottle 1004 with a square-shaped venting conduit 1006 extending into the bottle 1004 and connected to the square orifice 1008 of the adaptor body 1002 and tubing 1010 connected to the circular orifice 1012 of the adaptor body 1002. One end 1014 of the venting conduit 1006 may project out from the adaptor. The tubing 1010 may be connected to an ocular lens 1016 that irrigates a patient's eye 1018.

A further exemplary embodiment of the first aspect of the disclosure may provide a method for irrigation of an eye. In some embodiments, the method may involve the above-disclosed system. The method may comprise inserting the above-disclosed adaptor into the open end of the above-disclosed bottle. The method may further comprise inserting the above-disclosed venting conduit and the above-disclosed tubing into the adaptor via the first and second passageways, respectively, wherein the tubing is connected to the above-disclosed ocular lens configured for irrigation of the eye. The method may further comprise flowing liquid from the bottle and into the tubing. And the method may comprise irrigating the eye via the ocular lens.

In some embodiments, the method may further comprise removing a portion of the venting conduit so as to shorten the length of the venting conduit such that it fits inside the bottle. In some embodiments, the bottle may be plastic. Alternatively, the bottle may be made of any other functional material. In some embodiments, the bottle may be a disposable water bottle. In some embodiments, the bottle may contain or have previously contained water. Alternatively, the bottle may not be disposable or contain or have previously contained water.

In some embodiments, the method may further comprise flowing water from the bottle to the ocular lens so as to irrigate the eye. In some embodiments, the method may further comprise removing debris from the eye via irrigation and/or washing at least one chemical from the eye via irrigation. In some embodiments, the adaptor may connect an IV tubing spike of the ocular lens with a bottle to facilitate irrigation from a water reservoir. The adaptor may be a small, inexpensive, leak-free connection to the IV tubing spike, which provides a universal fit to disposable bottles or vessels. In some embodiments, the spike may be configured to extend through the above-disclosed second passageway.

In some embodiments, the above-disclosed method may comprise inserting an adaptor into an open end of a bottle, wherein the adaptor comprises a resilient, frustoconical body configured to be at least partially inserted into an open end of a bottle while forming a seal with a surface of the bottle but wherein the adaptor body either does not define first and second passageways or, alternatively, defines first and second passageways of different shapes and configurations than disclosed above.

### Methods

A universal adapter to facilitate emergency ocular irrigation in austere environments using the ocular lens of the '340 patent as an emergency eye irrigation device was developed through collaboration between an optometrist and a physician at the USU School of Medicine and engineers at the USU 4D Bio³ Center for Biotechnology. The adapter between the IV tubing of the ocular lens of the '340 patent and a disposable water bottle was designed to be small, inexpensive, provide a leak-free connection to the IV tubing sheath, and provide a universal fit to all disposable water bottles.

An initial design iteration included a screwcap adapter for a standard water bottle using Computer Aided Design (CAD) software that was 3D printed on a FormLabs Form 3B resin printer using biocompatible BioMed Clear resin. Because of inconsistent screwcap threads across different water bottle brands, the screwcap design was replaced with an internal stopper design, inspired by an Erlenmeyer flask stopper. The design includes an orifice for insertion of the IV spike that attaches the liquid reservoir to tubing. It was printed on an Ultimaker S5 printer that has been previously deployed in an austere Arctic environment. A third and final iteration was developed to include a 3D printed venting conduit to allow liquid to flow out of the bottle at a sufficient flow rate and was also printed on the Ultimaker S5.

Testing was completed to determine the fit of the adapter with bottles of various sizes, shape, and volume. Liquid flowrate from the water bottle was tested to ensure appropriate delivery of irrigating solution to the eye.

### Results

The initial screwcap design did not provide a universal adapter because of slight differences in cap thread design and dimensions between disposable water bottles. Additionally, the hard resin resulted in a brittle part that could be broken when carried in a battlefield medical first aid kit.

The internal stopper design provided a watertight fit across a range of bottle types. The material selected to print the stopper adapter was a thermoplastic polyurethane (Ultimaker TPU 95A) that provided both rigidity for insertion and flexibility for secure insertion and liquid seal. The initial stopper design did not allow air to enter into the bottle to replace water flowing into the IV tubing and ocular lens of the '340 patent. This caused water to quickly cease flowing into the IV tube once a vacuum had built up inside the water bottle. Therefore, a venting conduit was added to the design to allow for air displacement of the liquid in the water bottle. With the addition of the venting conduit, water flowed continuously into the IV tubing and was only limited by the IV tubing's flow regulator. The rigid venting conduit was printed using inexpensive polyethylene terephthalate glycol (PETG), although many other materials could have been used. The materials used in this design iteration are durable and the stopper design allows for accommodation of a wide variety of bottle types and sizes that may be present in resource constrained environments. This universal water bottle adapter device allows for leveraging supplies already carried by service members to reduce the weight and burden on deploying troops. The universal water bottle adapter device can be printed in less than 1 hour. The CAD files are available in an easily accessible format and can be printed prior to deployment or using an onsite printer.

In some embodiments, a step-wise use of the adaptor is discussed below:
Step 1: A venting conduit may include frangible portions spaced apart from one another and can be adjusted to match the length of the water bottle.
Step 2: The venting conduit may be inserted into the adaptor, along with the IV tubing spike of the ocular lens of the '340 patent. The adaptor may then be secured in the opening of the water bottle.
Step 3: The water bottle may then be inverted and a controllable valve opened to allow water to flow through the ocular lens.
Step 4: The ocular lens may be inserted and irrigation/lavage commenced.

In some embodiments, the venting conduit may be snappable to accommodate various bottle sizes. Frangible portions spaced apart from one another may allow for the venting conduit to be easily snapped at various locations, and the location of the frangible portions may be determined by the length of various common bottles. The venting conduit may be adjustable to accommodate various bottle sizes or, alternatively, may be pushed in a variable amount to accommodate any water bottle size.

In some embodiments, the disclosed adaptor may be employed for delivery of tuminescent anesthesia. In particular, lidocaine (0.05-0.10%) may be dissolved in a water bottle and salts (i.e., sodium chloride) added to obtain an isotonic solution. An antibiotic (i.e., 0.9-1.1% benzyl alcohol) may be included as an antibiotic preservative in bacteriostatic water to ensure sterility of water. The water bottle may be connected to the disclosed adaptor along with IV tubing and infusion commenced.

In some embodiments, the disclosed adaptor may be employed for delivery of water-soluble drugs intravenously. In particular, drug may be dissolved in a water bottle and salts (i.e., sodium chloride) added to obtain an isotonic solution. Examples of potential drugs for dissolution include sargramostim, anticibrinoylic agents (e.g., aminocaproic acid, tranexamic acid), analgesics (e.g., ketamine, acetaminophen, fentanyl), and antibotics, such as cefalozin, clindamycin phosphate, and ceftobiprole medocaril. An antibiotic (i.e., 0.9-1.1% benzyl alcohol) may be included as an antibiotic preservative in bacteriostatic water to ensure sterility of water. The water bottle may be connected to the disclosed adaptor along with IV tubing and infusion commenced.

In some embodiments, the disclosed adaptor may be employed for hemorragic shock treatment. In particular, the disclosed adaptor may be employed for delivery of fluids intravenously to replace lost blood volume. In particular, salts (e.g., sodium chloride, Ringer's lactate) may be added to create an isotonic solution. An antibiotic (i.e., 0.9% benzyl alcohol) may be included as an antibiotic preservative in bacteriostatic water to ensure sterility of water. The water bottle may be connected to the disclosed adaptor along with IV tubing and infusion commenced.

Other potential applications of the disclosed adaptor include reconstitution of a lyophilized plasma, rehydration of food, connecting water bottles to water filters for emergencies, or other eye lavage techniques.

### Conclusion

A universal water bottle adapter was developed as a cost effective and user-friendly option for rapid initiation of irrigation to injured eyes in a battlefield setting. The design allows for a watertight seal, easy insertion of the IV spike included in an emergency eye wash kit, and intuitive use for a hands-free eye irrigation in the critical window following ocular injury. The CAD files developed can be transferred and used to rapidly 3D print this device at or near the point of need. Implementation of the water bottle adapter will be field tested during an Ocular Trauma course offered at USU.

1. An adaptor comprising:
a resilient, frustoconical body configured to be at least partially inserted into an opening at a first end of a bottle while forming a seal with an inner peripheral surface of the opening,
the body defining first and second passageways extending through the body from an upper surface of the body to a lower surface of the body,
the first passageway having a generally square-shaped cross-section along at least a portion of a length of the first passageway, and
the second passageway having a generally circular-shaped cross-section along at least a portion of a length of the second passageway.

2. The adaptor of claim 1, wherein the body comprises thermoplastic polyurethane.

3. The adaptor of claim 1, wherein the first and second passageways are configured to receive a venting conduit and a tubing, respectively.

4. A system for use in delivering a liquid for treatment of an individual, the system comprising:
the adaptor of claim 3,
the venting conduit, and
the tubing,
wherein the venting conduit has a generally rectangular cross-section along at least a portion of the length of the venting conduit.

5. The system of claim 4, wherein the venting conduit has a plurality of frangible portions spaced apart from one another along the length of the venting conduit, the frangible portions being configured to permit a portion of the venting conduit to be removed from a remainder of the venting conduit so as to shorten the length of the venting conduit.

6. The system of claim 4, further comprising a bottle, wherein the bottle contains a liquid used for ocular irrigation for treatment of an eye.

7. The system of claim 6, wherein the venting conduit extends through the first passageway and protrudes into the bottle such that an end of the venting conduit is proximate to a closed end of the bottle opposite from the first end of the bottle.

8. The system of claim 4, wherein the tubing includes a spike configured to extend at least part way through the second passageway.

9. The system of claim 4, wherein the venting conduit comprises polyethylene terephthalate glycol.

10. The system of claim 4, wherein the tubing is connected to an ocular lens configured to irrigate an eye.

11. The system of claim 10, wherein the ocular lens is cup-shaped and defines an inner portion configured to cover a cornea and an outer portion defining a peripheral rim.

12. A method for irrigation of an eye, the method involving the system of claim 10, and comprising:
inserting the adaptor into the opening at the first end of the bottle;
inserting the venting conduit and the tubing into the adaptor through the first and second passageways, respectively,
wherein the tubing is connected to the ocular lens configured for irrigation of the eye;
flowing liquid from the bottle and into the tubing;
and
irrigating the eye with the liquid via the ocular lens.

13. The method of claim 12, further comprising removing a portion of the venting conduit so as to shorten the length of the venting conduit such that it fits inside the bottle.

14. The method of claim 12, wherein the bottle is a plastic bottle.

15. The method of claim 12, wherein the bottle is a disposable water bottle.

16. The method of claim 12, wherein the bottle contains or previously contained water.

17. The method of claim 12, wherein, in the flowing step, water flows from the bottle to the ocular lens so as to irrigate the eye.

18. The method of claim 12, wherein the irrigating step removes debris from the eye.

19. The method of claim 12, wherein the irrigating step washes at least one chemical from the eye.

20. A method for irrigation of an eye, the method comprising:
inserting an adaptor into an open end of a bottle, the adaptor comprising a resilient, frustoconical body configured to be at least partially inserted into the open end of the bottle while forming a seal with an inner peripheral surface of the open end of the bottle;
inserting a venting conduit and tubing into the adaptor, wherein the tubing is connected to an ocular lens configured for irrigation of the eye;
flowing liquid from the bottle and into the tubing;
and
irrigating the eye with the liquid via the ocular lens.

21-27. (canceled)
